(19) United States
(12) Patent Application Publication
(10) Pub. No.: US 2011/0159497 A1
(43) Pub. Date: Jun. 30, 2011

(54) FREEZE-DRIED COMPOSITIONS FOR CARRYING OUT PCR AND OTHER BIOCHEMICAL REACTIONS

(75) Inventors: Martin Alan Lee, Wiltshire (GB); Jennifer Mitchell, Ellesmere Port (GB); Emma Henderson, Wiltshire (GB)

(73) Assignee: ENIGMA DIAGNOSTICS LIMITED, Wiltshire (GB)

(21) Appl. No.: 12/664,183

(22) PCT Filed: Jun. 16, 2008
(86) PCT No.: PCT/GB2008/002036

§ 371 Date: Jun. 23, 2010

(30) Foreign Application Priority Data
Jun. 16, 2007 (GB) 0711683.3

(51) Int. Cl. C12Q 1/68

(52) U.S. Cl. 435/6.12

(57) ABSTRACT

A composition for carrying out a chemical or biochemical reaction, said composition being in a freeze-dried form and comprising (i) a set of reagents comprising at least some of the chemical or biochemical reagents necessary for conducting said chemical or biochemical reaction, (ii) a glass forming agent, (iii) a stabilising agent therefore and (iv) fish gelatine. In particular compositions for carrying out PCR are foreseen. Kits comprising these compositions and methods for using them form a further aspect of the invention.

The present invention relates to compositions comprising test reagents for use in chemical or biochemical reactions such as the polymerase chain reaction and to methods for preparing these.

Various preparations are available which provide for predetermined mixtures of reagents which are routinely used together. For instance, the widely used polymerase chain reaction (PCR) (including reverse transcriptase (RT)-PCR) utilises a range of standard reagents including salts such as magnesium chloride (MgCl₂) and potassium chloride, a polymerase enzyme such as Taq polymerase, buffers such as Tris-HCl, and nucleotides required for an amplification of a nucleic acid. Such preparations are available for example as “ready-to-go PCR beads” from Amersham BioSciences (UK) or Pharmacia.

Generally these are prepared by freeze drying methods, which are conducted in the presence of glass-forming agents and stabilisers for the structures formed as well as optionally fillers (see for example U.S. Pat. No. 5,250,429, U.S. Pat. No. 5,763,157 and EP-0726310) although other preparation types such as those which use wax carriers are also known (see for example U.S. Pat. No. 5,599,660).

These provide a convenient and readily available means for laboratories to conduct PCR reactions of their choice, when required. Generally, the specific reagents which tailor a PCR to the particular target, such as the primers and any probes required for example for use in connection with a Real-time PCR, are added on site, for example in the laboratory.

However, in many cases, in particular in the diagnostics field, the targets are the same in many cases, and therefore the inclusion of probes and primers into the bead, so that the bead becomes assay specific is desirable for ease of use.

A problem with all such beads and preparations is that the components do not always remain stable over long periods of time. As the nature of assays becomes more complex, further reagents including reagents that may include relatively sensitive chemical moieties such as labels and in particular optical labels such as fluorescent labels or dyes may be required to be added. These in particular are used for conducting assays in “real-time”. The sensitive moieties are frequently attached to olignonucleotides which are designed to act as probes or labelled primers. These will hybridise to amplified nucleic acids during the course of the PCR. The fate of the probes during the course of the PCR and changes in the associated signal from the label is used in various ways to monitor the progress of the PCR.

However, the presence of such moieties exacerbates the problems associated with the stability of the compositions.

Gelatine has previously been suggested in freeze-dried compositions as possible carrier proteins in complex multi-bead assay systems (see US2006/0066399) and in particular to enhance the effects of anti-freeze proteins (see WO2005/076908).

The applicants have produced more stable freeze-dried compositions.

The present invention provides a composition for carrying out a chemical or biochemical reaction, said composition being in a freeze-dried form and comprising (i) a set of reagents comprising at least some of the chemical or biochemical reagents necessary for conducting said chemical or biochemical reaction, (ii) a glass forming agent, (iii) a stabilising agent therefore and (iv) fish gelatine.

Compositions prepared in accordance with the invention are stable for prolonged periods, even in the absence of anti-freeze proteins. Furthermore, the fish gelatine in particular does not inhibit the reactivity of the composition. A single solid composition such as a bead or cake system may generally be prepared which is economical and easy-to-use.

When a composition is freeze-dried in the presence of a glass-forming reagent (ii), it generally forms a “cake” type 3-dimensional structure. This structure is supported by the inclusion of a suitable stabiliser (iii) for the cake structure, and so this is a further component of the mixture. However, the applicants have found that gelatine specifically from a fish source produces significant advantages in terms of stability of the composition, possibly by further stabilising the cake structure. In addition, it does not, unlike some gelatines, for example, those obtained from bovine, pig or seaweed (carrageenan) sources) appear to inhibit many complex chemical or biochemical reactions, in particular the polymerase chain reaction, such as a polymerase chain reaction carried out in real-time. However, the compositions may also be suitable for use in other assays or reactions, in particular those which rely upon the use of enzymes to effect the procedures such as nucleic acid sequencing reactions, other nucleic acid amplification reactions (including the ligase chain reaction (LCR), strand displacement amplification (SDA), transcription-mediated amplification (TMA), loop-mediated isothermal amplification (LAMP), rolling circle DNA amplification, multiplex ligation-dependent probe amplification (MLPA) and multiple displacement amplification.)

The fish gelatine is suitably included in the composition in an amount of about from 0.0001%-0.02% w/w, for example about 0.0025%-0.01% w/w, for instance at about 0.001-0.01% w/w, and in particular at about 0.006% w/w.

Fish gelatine, like other animal gelatines, may be obtained for example from the skin of the animal. A common source of fish gelatine is cod. Fish gelatine is a water-soluble protein. An aqueous solution of fish gelatine is generally liquid at room temperature, whereas gelatine from other animal sources is solid.

The precise composition of the fish gelatine will vary depending upon factors such as the source, but in general, it comprises a protein comprising a chain of for example 20 amino acids. The molecular weight of fish gelatine generally falls within the range of from 30,000 to 60,000. Fish gelatine can be obtained commercially in pure form, or it may be isolated from fish skin using conventional methods.

Suitable glass-forming reagents include sugars, in particular a non-reducing sugar, for example, trehalose, sucrose or mannose. This is suitably present in the composition in an amount such that it represents from about 1-10% w/w and suitably about 5% w/w in the final composition.

Examples of suitable stabilisers that may be included in the composition include polymeric compounds such as polyethylene glycol (PEG), polyvinylpyrrolidine (PVP) and or polysaccharides such as Ficoll or Dextran.

The set of reagents (i) above will be selected depending upon the particular nature of the chemical or biochemical reaction being effected. They may include reactions carried out on multiple or repeated occasions such as diagnostic tests, screening tests, nucleic acid amplification reactions, sequencing reactions etc.

In a particular embodiment, the set of reagents is a set of reagents which is specifically adapted to carry out a polymerase chain reaction (PCR). In this case, item (i) will generally comprise a polymerase capable of extending a primer when adhered to a template nucleic acid sequence during a polymerase chain reaction. The template nucleic acid may be a DNA or, in the case of RT-PCR, an RNA sequence.

Suitably the set of reagents of item (i) above further comprises a buffer, salt (such as magnesium or manganese salts for example magnesium or manganese halides), one or more primers and nucleotides required to construct the extension to the primer(s) which are required to effect a polymerase chain reaction to amplify a target DNA sequence. However, it is possible that one or more of these elements may be missing in particular where these elements can be readily added later, for example in a rehydration buffer used to reconstitute the dried composition ready for use. In particular, the necessary salts may be added in this way and so the set of reagents of (i) may omit the salts. Where this is done, the composition may be supplied in the form of a kit with rehydration buffer, containing the necessary salt supplements.

The composition may further comprise a labelled oligonucleotide useful in monitoring the progress of a polymerase chain reaction in real time. Also as used herein, the expression “real-time” means that the polymerase chain reaction can be monitored as it progresses and without halting or opening the reaction vessel. By monitoring how the amplification occurs and in particular at which cycles exponential increase in amplicon becomes significant allows the amount of target nucleic acid present in the sample being subject to the PCR to be quantitated as is well known and understood in the art.

The amounts of the various components included in the composition will vary depending upon factors such as the precise nature of the particular component, the nature of the PCR which it is intended should be conducted etc. However, this will be determinable in each case using established protocols and procedures as would be understood in the art.

Suitable labelled oligonucleotides are any of the labelled probes or labelled primers which may be used in the monitoring of polymerase chain reactions in real time. Thus in a particular embodiment they will comprise probes which are capable of hybridising to the amplified nucleic acid sequence and which carry labels in particular, optical labels such as fluorescent labels which provide a signal which varies in accordance with the progress of the PCR.

Thus for probes intended to be utilised in a TAQMAN™ assay, for example, they will generally comprise a probe which carries two labels, one of which is able to act as a donor of energy and particularly fluorescent energy, and one of which is able to act as an acceptor of that energy or “quencher”. Whilst the probe is intact, these labels are held in close proximity to each other so that interaction of energy occurs. In the case of fluorescent labels, this is known as flurorescent energy transfer (FET) or fluorescent resonant energy transfer (FRET).

The probes are designed to bind to a specific region on one strand of a template nucleic acid. Following annealing of the PCR primer to this strand, Tag enzyme extends the DNA with 5′ to 3′ polymerase activity. Taq enzyme also exhibits 5′ to 3′ exonuclease activity. TaqMan™ probes are protected at the 3′ end by phosphorylation to prevent them from priming Tag extension. If the TaqMan™ probe is hybridised to the product strand, an extending Taq molecule will hydrolyse the probe, liberating the donor from acceptor. This means that the interaction between the donor and the acceptor is broken, so the signal from each, changes, and this change can be used as the basis of detection. The signal in this instance is cumulative, the concentration of free donor and acceptor molecules increasing with each cycle of the amplification reaction.

Hybridisation probes are available in a number of forms and these may also be included in the compositions. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated, so FRET does not occur, and this forms the basis of detection during a polymerase chain reaction.

Pairs of labelled oligonucleotides may also be used as probes in the detection of a polymerase chain reaction. These hybridise in close proximity on a PCR product strand-bringing donor and acceptor molecules together so that FRET can occur. Enhanced FRET is the basis of detection. Methods of this type are described for example in European Patent Application No. 0912760 the entire content of which is incorporated herein by reference. Variants of this type include using a labelled amplification primer with a single adjacent probe.

WO 99/28500 (the entire content of which is incorporated herein by reference) describes a very successful assay for detecting the presence of a target nucleic acid sequence in a sample. In this method, a DNA duplex binding agent and a probe specific for said target sequence, is added to the sample. The probe comprises a reactive molecule able to absorb fluorescence from or donate fluorescent energy to said DNA duplex binding agent. This mixture is then subjected to an amplification reaction in which target nucleic acid is amplified, and conditions are induced either during or after the amplification process in which the probe hybridises to the target sequence. Fluorescence from said sample is monitored.

Thus, compositions adapted for use in this assay, known as ResonSense™ may also be prepared. In this instance, the composition will suitably further comprise a DNA duplex binding agent such as an intercalating dye.

An alternative form of this assay, which utilises a DNA duplex binding agent which can absorb fluorescent energy from the fluorescent label on the probe but which does not emit visible light, is described in WO2004/033726, the entire content of which is incorporated herein by reference.

In general, all probes used in these types of assays are blocked to extension at the 3′end for example by phosphorylation, or by having a label directly attached at the 3′ hydroxyl group. This prevents the probe from acting as a secondary primer, and being extended during the PCR, and so eliminates interfering products.

The amounts of probe utilized in any particular composition will vary depending upon factors such as whether it is used up or hydrolysed during the PCR, as well as the nature of the signaling system. These would be understood by the skilled person. Generally however, the amount of each probe added to a composition will be sufficient to ensure that the concentration of probe in the final composition is between 0.05 μM to 1 μM, for example at about 0.2 μM.

Other real-time assays utilize labeled primers in order to provide a monitoring system. Some of these primers may include a self-probing “tail” and are known as “Scorpion” primers. A labelled probe is linked to a DNA sequence which acts as a primer to the reaction by way of a “blocking group” which is suitably a chemical linker or non-amplifiable monomer such as hexethylene glycol and which prevents an extension reaction amplifying the probe region of the olignucleotide.

Probe/primer combinations of this general type are well known as “Scorpions” and these are described for instance in WO 99/66071. The Scorpion may along its length comprise a donor/quencher pair so that FRET signalling is possible as described above.

A further class of real-time probes called LUX™ (light upon extension) fluorogenic primers are also available. These are “hairpin” like probes, similar to the molecular beacons as described above. However, LUX primers adopt a stem-loop structure in solution, and like Scorpion probes, LUX primers are intended for use as PCR primers. They do not contain a quencher moiety as they are fluorescent oligonucleotides which are designed to self-quench based on sequence context. LUX primers quench when free in solution, fluoresce weakly when denatured, and emit light strongly when incorporated into DNA. These also may be included in the compositions of the invention.

The polymerase included in the set of reagents (i) is selected so that it is useful in conducting the desired “real-time” assay. Thus for assays such as TAQMAN™, where hydrolysis of the probe is essential in order to initiate a detectable signal, a polymerase having a high level of 5′-3′ exonuclease activity is suitably employed, whereas for assays such as Resonsence™ assays, where probe hybridization is employed, such activity may be low or absent. In addition, the probe may be designed to favour hydrolysis or hybridization by means of the enzyme. For example, a probe which is designed close to the primer on the target sequence will be more susceptible to any 5′-3′ exonuclease activity of the polymerase than a probe which binds further downstream of the primer sequence.

The polymerase is suitably a thermostable polymerase which will operate and withstand the elevated temperatures needed for conducting a polymerase chain reaction. The amount of polymerase added should be sufficient to effect a PCR reaction, as is understood in the art. Typically, the amount of polymerase added will be sufficient to provide a concentration of from 0.02 to 1.0 U/μl composition and typically about 0.025 U/μl.

Suitably, the composition may further comprise reagents which are used in ensuring that the polymerase chain reaction does not start prematurely. So called “Hot-Start” PCR may be effected by various methods.

The problem addressed by a “Hot-Start” PCR arises because a successful PCR relies on the sequence of steps, denaturation, annealing and extension, occurring in a very precise order and at the precise temperature required for the operation of that step. A problem arises when reagents are mixed together, even for short periods of time, at different temperatures, for example prior to the start of the reaction. Primers may interact with nucleic acid template, resulting in primer extension of the template. This can lead to a reduction in the overall yield of the desired product as well as the production of non-specific products.

Initial attempts to overcome the problem used a wax barrier to separate the various PCR reagents from each other in a test tube (see for example U.S. Pat. No. 5,565,339). The wax melted as the reaction mixture was heated to the initial denaturation temperature, allowing the reagents to mix together at the last possible moment, so that the possibility of side-reactions was minimized, and this gave rise to the expression “Hot Start”.

Other chemical methods for achieving the suppression of side-reactions have been attempted. For example, U.S. Pat. No. 5,677,152 describes a method in which the DNA polymerase is chemically modified to ensure that it only becomes active at elevated temperatures. In order to effect this method, it is necessary only to include an appropriately modified DNA polymerase in item (i) above.

In another embodiment, a monoclonal antibody to Thermus aquaticus (Taq) DNA polymerase such as the anti-Taq DNA polymerase antibody available from Sigma, is including into the composition. The antibody binds to the enzyme, so as to inactivate it, at ambient temperature. However, the antibody denatures and dissociates from the enzyme at elevated temperatures used during the amplification cycles and so the enzyme becomes active.

The relative amount of any anti-Taq antibody included in the composition is suitably sufficient to ensure that it is able to fulfill the function of inhibiting the Taq enzyme until it is required. Generally therefore an excess of anti-Taq antibody as compared to Taq enzyme will be used. Thus for example for every unit of Taq enzyme in the composition, at least 1.5 and preferably at least 2 units of anti-Taq antibody will be included. Anti-Taq antibody is usually sold by the μg and the concentration is very dependant upon the source and quality of the antibody as well as the nature of the assay. Too much antibody may be detrimental and can actually cause more primer dimmer in some assays. However, the precise amount of Taq antibody will be determined in accordance with usual practice and will typically be in the range of 0.001 to 0.004 μg/μl final reaction mixture.

Yet another Hot-Start methodology involving the use of a combination of an inhibitory amount of a pyrophosphate salt to prevent primer extension taking place, and a pyrophosphatase enzyme which digests this pyrophosphate at elevated temperatures, to allow the PCR to progress is described in WO 02/088387, the entire content of which is incorporated herein by reference.

In this case, the pyrophosphate salt and the pyrophosphatase enzyme may be included as further components of the composition of the invention.

An optional additional component of the composition of the invention is an anti-oxidant and/or anti-maillard agent. A particular example of such a reagent is threonine such as L-threonine although others may be used. These eliminate any oxygen produced and therefore assist in the stabilisation of the reaction mixture. They are suitably added in an amount which does not affect the pH of the composition, as determined by the buffer, which is generally between 8.3 and 9, for instance between 8.5 and 8.8. The amount of anti-oxidant which can therefore be added will depend upon the nature of the anti-oxidant itself. For example, for threonine it may be present in the composition in an amount of from 2-10 mM, for example at about 2.5 mM.

The precise selection of stabiliser (iii) will depend to some extent on the particular assay intended to be carried out using the final composition and this can be tested using routine methods. For example, it has been found that dextran is less preferred when the composition includes DNA duplex binding agents and labelled probes intended and is intended to be used to conduct a ResonSense™ assay as described above. However, PEG is a particularly suitable stabiliser for most of these compositions. Stabiliser is suitably added in an amount such that it represents from about 1-3% w/w in the final composition.

As discussed above, the set of reagents of item (i) may comprise components such as buffers, primers, nucleotides and optionally also salts, in the amounts which are generally understood for the preparation of PCR reaction mixtures. Primers are suitably present in excess and this is typically achieved by including sufficient primers to ensure that the concentration of each primer in the final composition is of the order of 0.1 μM to 1 μM.

Compositions of the invention may further comprise an RNase inhibitor. The applicants have surprisingly found that addition of RNase inhibitors has a stabilising effect on the composition, even where the composition contains no RNA elements or is intended for use in amplification reactions in which RNA is involved, such as RT-PCR. Its addition improves the stability of the composition, even over prolonged time periods, at the end of which, the composition is still able to operate in an effective manner when used in real-time PCR methods.

Without being constrained by theory, it is possible that they are assisting in preventing attack of the probe by the polymerase or otherwise controlling the activity of the polymerase, for reasons that are not understood. The number of units of RNase inhibitor (for example the RNase inhibitor available commercially as RI Out available from Invitrogen), is suitably should be sufficient to control the activity of the polymerase in the composition. Thus generally the number of units of RNase inhibitor will be of the same general order or preferably be higher than the amount of polymerase present in the composition to ensure effective inhibition. For example, where 0.05 U/μl polymerase is included in a composition, this will contain from 0.04 to 0.1 U/μl RNase inhibitor.

In a particular embodiment, a blocking compound, as is conventional in PCR reaction mixtures, may be included in the composition. The blocking compound is believed to function by preventing inhibition of the PCR by interaction with the vessel walls, for example by preventing leaching of metals or sequestering any metals which may leach from the walls in the course of the reaction. The nature of the blocking compound will depend upon the nature of the vessel into which it is intended that the reaction should be conducted.

Particular examples of blocking compounds are glass coating or glass blocking compounds such as bovine serum albumin (BSA) either alone or in combination with other blocking materials such as gelatine. Although, gelatine used as a blocking agent may be obtained from a variety of sources including bovine, pig, seaweed (carrageenan), the fish gelatine which is an element of the composition of the invention may provide a useful additional blocking effect.

Blocking agents are suitably included in effective amounts which will depending upon the particular compound selected. However, for BSA for instance, the amount is suitably sufficient to provide from 0.1 to 1 mg/ml and preferably about 0.25 mg/ml in the final composition.

Further components may be included in the composition as would be understood in the PCR art. These might include sequences used as internal controls as well as primers for amplifying these sequences and signalling systems such as those outlined above for detecting amplification of the internal control sequences.

Compositions of the invention are suitably prepared by mixing together the required components as described above to form a composition, and adding water, preferably sterile water which been treated with diethyl pyrocarbonate (DEPC)to the composition to allow for mixing, for example by adding at least equivalent volume and preferably from 1-1.5 times the volume of the composition. The thus formed mixture is, if necessary dispensed into suitable aliquots each of which contains sufficient material for a PCR in an individual reaction pot, and then subjected to a freeze drying process. If freeze drying does not take place immediately, the final mixture is suitably stored at low temperatures, for example on ice, or in a freezer if the delay is prolonged beyond about 0.5 hours, until freeze drying takes place.

The freeze-drying protocol used will depend to some extent upon the particular composition being dried and will be determined in each case using routine procedures. Typically, the composition will be subject to a freezing step in which it is cooled to a low temperature for example from about −20° C. to −60° C. and generally at about −40° C. at a pressure of from 300-400 torr, and held at this temperature for a sufficient period of time to ensure that complete freezing occurs.

The pressure is then reduced to an appropriate level depending upon the particular freeze-dryer used. Some may operate a pressures as low as 6 Mtorr but for current purposes, pressures of from 10 to 100 mTorr may be suitable to allow the water to sublimate. Suitably then the composition is brought gradually back up to room temperature under reduced pressure, before the vacuum is released to minimise condensation effects. Optionally, the vacuum is released in the presence of an inert atmosphere such a nitrogen, so that the product is maintained in an inert environment. This also prevents moisture ingress.

Freeze-dried product obtained in this way, it is suitably packaged immediately for example in foil wrappers, to minimise the contamination risk. If the composition is contained within containers such as reagent pots, these are suitably sealed before the vacuum is released.

Care needs to be taken to ensure that all reagents utilised in the composition do not contain materials or contaminants which could inhibit or prevent freeze drying in the levels in which they are found. Thus for example, it may be necessary to remove substances such as glycerol which are sometimes included in commercially available enzymes such as polymerases, reverse transcriptase polymerases and RNase inhibitors, and or to reduce the levels of substances such as dimethyl sulphoxide (DMSO) which may be found in intercalating dyes which may be used as DNA duplex binding agents.

Compositions as described above have been found to be stable for extended periods of time, including up to 3 months, at the end of which, no activity loss at all was seen.

Methods for forming compositions described above form a further aspect of the invention. In a particular embodiment, the invention provides a method for preparing a freeze dried composition, which comprises mixing together at least items (i) to (iv) above and freeze drying the resultant mixture.

In use the compositions of the invention are hydrated using conventional methods, for example using a rehydration buffer and then subject to the appropriate chemical or biochemical reaction. Generally, the composition will be mixed with a chemical or biochemical sample before the reaction is conducted. For example, in the case of a polymerase chain reaction, the reaction mixture is combined with a sample which contains or is suspected of containing a target nucleic acid, and the final mixture subjected to PCR conditions, with monitoring in real-time as required, if the appropriate signalling reagents are present. Such methods form a further aspect of the invention.

The invention will now be particular described by way of example, with reference to the accompanying drawings in which:

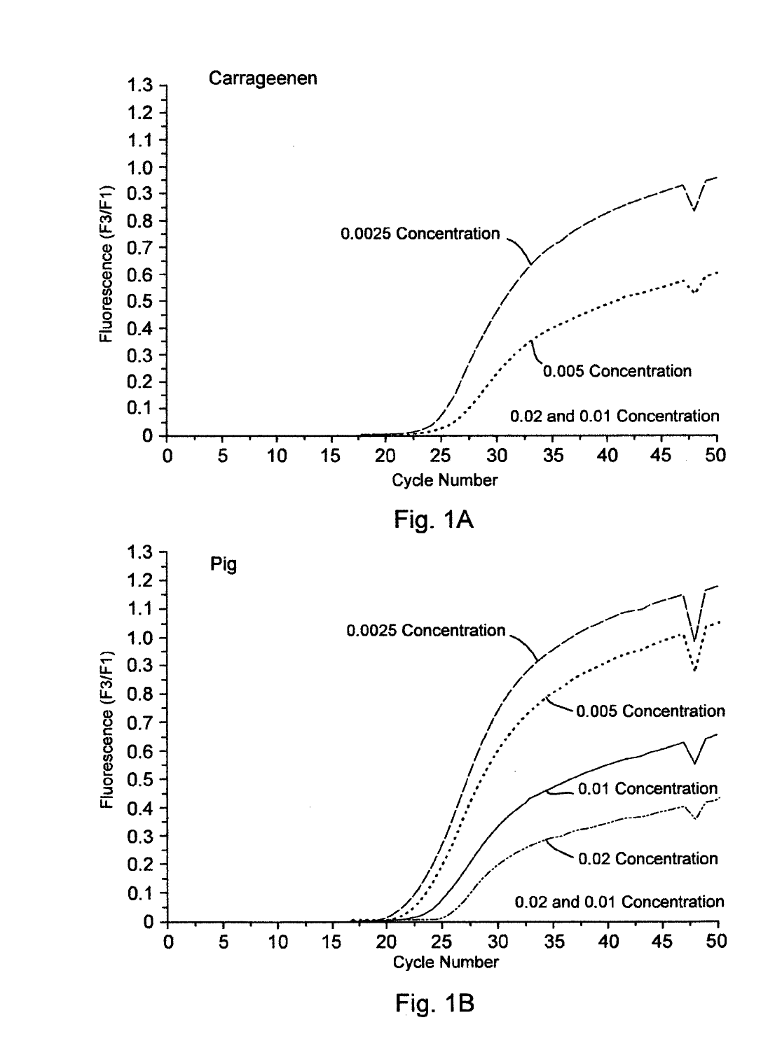

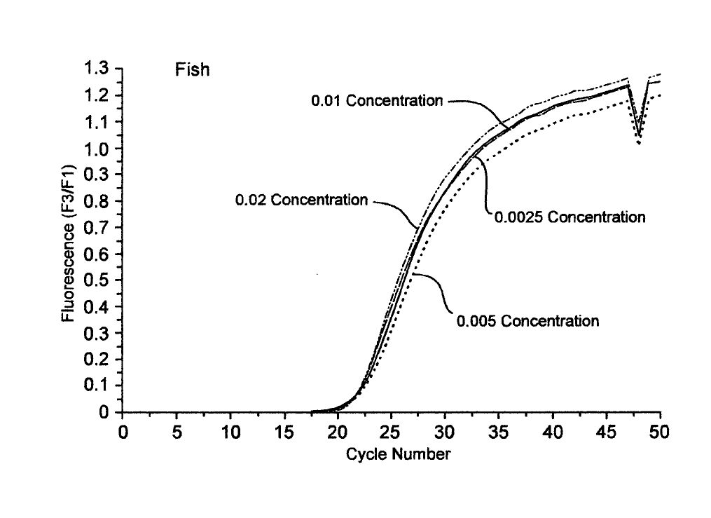

FIG. 1 shows the result of conducting a real-time PCR assay using dual hybridisation probes in the presence of various concentrations of (A) carrageenan, (B) porcine gelatine and (c) fish gelatine.

### EXAMPLE 1

### Preparation of Composition for Conducting LUX™ Assay for Detecting Bacillus subtilis var. globigii (BG) DNA and Spores

A composition comprising the reagents listed in Table 2 was prepared. Primers were designed using conventional primer design software to amplify a BG specific DNA sequence.

TABLE 2

| | | | Final | |
|---|---|---|---|---|
| | | | concentration | |
| Reagent | Conc. | | in reaction | |
| RI Out ribonuclease | 5 | U/μl filtered | 0.1 | U/μl |
| inhibitor* | | | | |
| Tris pH 8.8 | 500 | mM | 50 | mM |
| BSA | 20 | mg/ml | 0.25 | mg/ml |
| MgCl₂ | 100 | mM | 3 | mM |
| dUTP mix | 2 | mM | 0.2 | mM |
| Fish gelatine | | | 0.006% | |
| LUX labelled primer | 10 | μM | 0.5 | μM |
| Reverse primer | 10 | μM | 0.5 | μM |
| Trehalose | 50% | w/v | 10% | |
| PEG 20,000 | 10% | w/v | 1% | |
| L-threonine | 400 | mM | 10 | mM |
| Taq antibody | 5 | U/μl filtered | 0.1 | U/μl |
| Taq polymerase | 5 | U/μl filtered | 0.05 | U/μl |

| | |
|---|---|
| Water | DEPC treated |

*Available from Invitrogen

Once these reagents had been combined in a reaction tube, it was stored on ice and dispensed in 50 μl aliquots into reagent pots which had been pre-chilled in a fridge within half an hour. These were then placed inside a freeze dryer (Virtice Advantage), which was set to carry out the program summarised in Table 3.

TABLE 3

| | | Time | Pressure | Ramp/ |
|---|---|---|---|---|
| Step | Temp ° C. | (min) | (Torr) | Hold |
| Thermal Treatment | | | | |
| 1 | +10 | 15 | 3-400 | H |
| 2 | −40 | 55 | 3-400 | R |
| 3 | −40 | 120 | 3-400 | H |
| Freeze, condenser, vac | 040 | 0 | 100 mTorr | |
| Primary Drying | | | | |
| 1 | −40 | 45 | 100 mTorr | H |
| 2 | +5 | 55 | 100 mTorr | R |
| 3 | +5 | 30 | 100 mTorr | H |
| 4 | +20 | 25 | 100 mTorr | R |
| 5 | +20 | 300 | 100 mTorr | H |
| 6 | +5 | 25 | 100 mTorr | R |
| 7 | +5 | 300 | 100 mTorr | H |
| 8 | +20 | 25 | 100 mTorr | R |
| 9 | +20 | 15 | 100 mTorr | H |
| 10 | +10 | 20 | 100 mTorr | R |
| 11 | +10 | 1000 | 100 mTorr | H |
| Secondary Drying | +27 | | | |
| | set point | | | |
| Post Heat Settings | +10 | 1000 | 100 mTorr | |

The pots were then removed from the freeze dryer and foil sealed immediately. They were stored at room temperature, and retained full activity when tested after 3 weeks.

### EXAMPLE 2

### Preparation of Composition for Conducting Scorpion™ Assay for Detecting CHL DNA

The procedure of Example 1 was generally followed, but in this case, a Scorpion™assay for the CHL DNA was prepared. A composition comprising the reagents listed in Table 3 was prepared

TABLE 3

| | | | Final | |
|---|---|---|---|---|
| | | | concentration | |
| Reagent | Conc. | | in reaction | |
| RI Out ribonuclease | 5 | U/μl filtered | 0.1 | U/μl |
| inhibitor | | | | |
| Tris pH 8.8 | 500 | mM | 50 | mM |
| BSA | 20 | mg/ml | 0.25 | mg/ml |
| MgCl₂ | 100 | mM | 3 | mM |
| dUTP mix | 2 | mM | 0.2 | mM |
| Fish gelatine | | | 0.006% | |
| Scorpion labelled primer | 10 | μM | 1 | μM |
| Reverse primer | 10 | μM | 1 | μM |
| Trehalose | 50% | w/v | 10% | |
| PEG 20,000 | 10% | w/v | 2% | |
| L-threonine | 400 | mM | 10 | mM |
| Taq antibody | 5 | U/μl filtered | 0.1 | U/μl |
| Taq polymerase | 5 | U/μl filtered | 0.05 | U/μl |

| | |
|---|---|
| Water | DEPC treated |

Once these reagents had been combined in a reaction tube, they were freeze dried as described above in Example 1.

### EXAMPLE 3

### Evaluation of Stability of Compositions for Conducting Dual Hybridisation Assay for Detecting Bacillus subtilis var. globigii (BG) DNA and Spores

In an initial experiment, three different gelatines were tested at four separate concentrations to see if they inhibited a real-time dual hybridisation assay. Compositions were prepared from the components listed in Table 4 together with each of fish gelatine, porcine gelatine and seaweed gelatine (carrageenan) at concentrations of 0.02%, 0.01%, 0.005% and 0.0025% w/w:

TABLE 4

| | | | Vol. (μl | | |
|---|---|---|---|---|---|
| | | | per 50 μ1 | Final | |
| | | | reaction | concentration | |
| Reagent | Conc. | | volume) | in reaction | |
| RI Out | 5 | U/μl filtered | 0.5 | 0.1 | U/μl |
| ribonuclease | | | | | |
| inhibitor | | | | | |
| Tris pH 8.8 | 500 | mM | 2.5 | 50 | mM |
| BSA | 20 | mg/ml | 0.3 | 0.25 | mg/ml |
| MgCl₂ | 100 | mM | 0.75 | 3 | mM |
| dUTP mix | 2 | mM | 2.5 | 0.2 | mM |
| Forward primer | 10 | μM | 2.5 | 1 | μM |
| Reverse primer | 10 | μM | 2.5 | 1 | μM |
| FAM labelled | 2 | μM | 2.5 | 0.2 | μM |
| Donor probe | | | | | |
| Cy5 labelled | 2 | μM | 2.5 | 0.2 | μM |
| acceptor probe | | | | | |
| Trehalose | 50% | w/v | 2.5 | 5% | |
| PEG 20,000 | 10% | w/v | 7.5 | 1% | |
| Taq antibody | 5 | U/μl filtered | 0.5 | 0.1 | U/μl |
| Taq polymerase | 5 | U/μl filtered | 0.25 | 0.025 | U/μl |

| | | |
|---|---|---|
| Water | DEPC treated | 23.8 |

Each was then subjected to a real-time PCR experiment in which a predetermined amount of BG DNA was included, and the results monitored. The results are shown in FIG. 1 where 1A is the results obtained with carrageenan, 1B shows the results of the porcine gelatine compositions and 1C shows the results of the fish gelatine containing compositions.

Although carrageenan was inhibitory at all concentrations, and porcine gelatine did appear to inhibit the result at least at some concentrations, fish gelatine appeared to have no inhibitory effects at any concentration.

Mixtures as described in Table 4 were prepared and each included either 0.0025% fish gelatine, 0.0025% porcine gelatine or carrageenan at a concentration of 0.0002% w/w. A control mixture was prepared without gelatine. These were then aliquoted into individual pots and each was then subjected to a freeze drying procedure generally as described in Example 1 and stored.

Batches of four pots from each sample type including the control were sampled at weekly intervals to assess the stability of the compositions. This was done by carrying out a real-time PCR assay for BG DNA. Foil covers were removed from the pots, and dH₂O (23 μ) was added to all pots to re-hydrate the freeze dried cake. The samples were then transferred into capillary vessels for use in a LightCycler™. At that point, further dH₂O (2 μl) was added to two of the pots of each sample type, (negative control) and DNA from BG extracted cells (2 μl ) added to the other two pots.

Each capilliary vessel was then capped, spun in a centrifuge (3000 rpm) for a few seconds, taken out and placed into a LightCycler carousel. Each was subjected to a PCR reaction with continuous monitoring in accordance with the following program:

| | |
|---|---|
| Initial denaturation : | 95° C.-2 minutes |
| 50 cycles of : | 95° C.-5 seconds |
| | 55° C.-20 seconds |
| | 74° C.-5 seconds |

Although both the fish and porcine gelatine samples initially showed high ct and fluorescence, comparable to the no gelatine control, they appeared to retain stability longer than the no gelatine control, and the fish gelatine maintained the highest ct levels for the longest time period.

1. A composition for carrying out a chemical or biochemical reaction, said composition being in a freeze-dried form and comprising (i) a set of reagents comprising at least some of the chemical or biochemical reagents necessary for conducting said chemical or biochemical reaction, (ii) a glass forming agent, (iii) a stabilising agent therefore and (iv) fish gelatine.

2. The composition of claim 1 wherein the fish gelatine is present in the composition in an amount from about 0.0001% to about 0.02% w/w.

3. The composition of claim 1 wherein the glass-forming reagent (ii) is a non-reducing sugar.

4. The composition of claim 1 wherein the stabiliser (iii) is selected from the group consisting of polyethylene glycol (PEG)-, polyvinylpyrrolidine (PVP) and polysaccharide.

5. The composition of claim 1 wherein the set of reagents (i) is a set of reagents which is specifically adapted to carry out a polymerase chain reaction (PCR)-.

6. The composition of claim 5 wherein the set of reagents comprises a polymerase capable of extending a primer when adhered to a template nucleic acid sequence during a polymerase chain reaction.

7. The composition of claim 5 wherein the set of reagents includes all of buffer, salts, primers and nucleotides suitable for conducting a polymerase chain reaction to amplify a DNA sequence.

8. The composition of claim 5 wherein the set of reagents includes buffer, primers and nucleotides suitable for conducting a polymerase chain reaction to amplify a DNA sequence.

9. The composition of claim 1 which further comprises a labelled oligonucleotide useful in monitoring the progress of a polymerase chain reaction in real time.

10. The composition of claim 9 wherein the labelled oligonucleotide is a probe which carries two labels, one of which is able to act as a donor of energy and one of which is able to act as an acceptor of that energy.

11. The composition of claim 9 wherein the labelled oligonucleotide is in the form of a molecular beacon.

12. The composition of claim 9 which comprises a pair of labelled probes one of which carries a label which is an energy donor and one of which carries a label which is able to accept energy from said energy donor, and wherein the probes hybridise in close proximity on a PCR product strand.

13. The composition of claim 9 wherein the composition further comprises a DNA duplex binding agent which is able to exchange energy with said labeled oligonucleotide.

14. The composition of claim 5 which further comprises one or more reagents able to control the initiation of the PCR reaction.

15. The composition of claim 14 wherein the reagent is an anti-Taq antibody.

16. The composition of claim 5 which further comprises an RNAse inhibitor.

17. The composition of claim 16 wherein the number of units of RNase inhibitor will be at least the same or higher than the amount of polymerase present in the composition.

18. The composition of claim 1 which further comprises an anti-oxidant and/or anti-maillard agent.

19. The composition of claim 1 which further comprises a blocking compound.

20. A method for preparing a composition comprising (i) a set of reagents comprising at least some of the chemical or biochemical reagents necessary for conducting a chemical or biochemical reaction, (ii) a glass forming agent, (iii) a stabilising agent therefore and (iv) fish gelatine, said method comprising mixing together the components of the composition and freeze drying the resultant mixture.

21. A kit comprising a composition comprising (i) a set of reagents comprising at least some of the chemical or biochemical reagents necessary for conducting a polymerase chain reaction, (ii) a glass forming agent, (iii) a stabilising agent therefore and (iv) fish gelatine; and a rehydration buffer.

22. The kit of claim 21 wherein the composition does not contain salts necessary for carrying out the reaction, and the rehydration buffer includes said salts.

23. A method for conducting a chemical or biochemical reaction which comprises hydrating a freeze-dried composition comprising (i) a set of reagents comprising at least some of the chemical or biochemical reagents necessary for conducting the chemical or biochemical reaction, (ii) a glass forming agent, (iii) a stabilising agent therefore and (iv) fish gelatine; and subjecting the hydrated composition to conditions under which the chemical or biochemical reaction will occur.
